(19) United States
(12) Patent Application Publication
(10) Pub. No.: US 2026/0232749 A1
(43) Pub. Date: Aug. 13, 2026

(54) Plant-Based Compositions And Methods For Modulation of Inflammatory Response Post Viral Infection

(71) Applicant: VDF Futureceuticals, Inc., Momence, IL (US)

(72) Inventors: Boris Nemzer, Momence, IL (US); Pietrzkowski Zbigniew, Momence, IL (US); John M Hunter, Momence, IL (US)

(73) Assignee: VDF Futureceuticals, Inc., Momence, IL (US)

(21) Appl. No.: 19/155,856

(22) PCT Filed: Feb. 14, 2024
(86) PCT No.: PCT/US24/15696

§ 371 Date: Aug. 12, 2025

Related U.S. Application Data
(60) Provisional application No. 63/484,981, filed on Feb. 14, 2023.

(51) Int. Cl. A61K 36/74; A61K 9/00; A61K 31/05; A61K 31/12; A61K 31/122; A61K 31/355; A61K 33/32; A61K 36/31; A61K 36/736; A61K 36/82; A61K 36/9066; A61P 37/06; A61P 39/06

(52) U.S. Cl. CPC A61K 36/742; A61K 9/0053; A61K 31/05; A61K 31/12; A61K 31/122; A61K 31/355; A61K 33/32; A61K 36/31; A61K 36/736; A61K 36/82; A61K 36/9066; A61P 37/06; A61P 39/06

(57) ABSTRACT

A polyphenol-rich composition is orally administered to alleviate a sign or symptom of a post-viral syndrome, and preferably post-COVID syndrome. Most typically, the composition comprises at least 50 wt % total catechins, at least 20 wt % total chlorogenic acids, and optionally up to 30 wt % total supplemental antioxidants. The composition may be formulated from a green coffee bean extract, a green tea extract, a turmeric extract, a tart cherry or extract thereof, a broccoli or extract thereof, and a kale or extract thereof. Notably, such compositions were effective in individuals post SARS-CoV2 infection to reduce proinflammatory cytokines and interleukins, increase NOHb, and to reduce reactive oxygen species due to mitochondrial dysfunction, iNOS activity, and NOX2 activity.

### CROSS REFERENCE TO RELATED APPLICATIONS

This application claims priority to our U.S. Provisional patent application with the Ser. No. 63/484,981, filed Feb. 14, 2023, incorporated by reference herein.

### FIELD OF THE INVENTION

The field of the invention is compositions and methods for nutritional supplements that alleviate signs or symptoms after a viral infection, and especially as it relates to post-SARS-CoV2 infection responses.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Over the course of the COVID-19 pandemic, hundreds of millions of people were infected worldwide, leaving a global burden for long-term care of COVID-19 survivors. Among other sequelae of SARS-CoV2 infection, viral exposure affects a variety of organ systems, even after complete clearance of the virus. For example, adverse effects of infection were reported to extend to the immune, the hematological, the pulmonary, the cardiovascular, the gastrointestinal, the hepatic, the renal, the skeletomuscular, and the nervous systems. But these lasting, damaging, effects persist even after other viral infections such as influenza, rhinovirus (RV), respiratory syncytial virus (RSV), and Middle Eastern Respiratory Syndrome (MERS).

In many infected individuals, viral clearance results in complete resolution of symptoms. However, a significant number of individuals continue to suffer from post-viral syndrome. It is commonly believed that the diverse adverse effects are precipitated by reduction-oxidation reaction (redox) imbalance of the endothelial system. A systemic imbalance is often distinguished by increased activity of ROS pathways (including phagocytic NADPH oxidase (NOX2), inducible nitric oxide synthase (iNOS), mitochondrial), and concomitant suppression of NOHb. Together, these four conditions are often associated with the release of excessive proinflammatory cytokines and chemokines, that result in cellular oxidative stress and feelings of illness commonly associated with viral insult. In more severe cases, high levels of proinflammatory cytokines (IL-6, IL-1 and TNF-α) with pleiotropic abilities were found to interact with their high-density receptors, immune cells, and vasculature, which then stimulate many processes involved with further activation of immune cells in response to changes in the vascular environment. The most common symptoms of which are fatigue and dyspnea, but also include cognitive and mental impairments, persistent pathological and chronic sub-acute inflammation.

An “imbalanced” endothelial system can result from various etiologies, including metabolic syndrome, cardiotoxicity and other chronic conditions, as well as acute bacterial, viral and environmental insult. However, there is only a small body of evidence that substantiates a way in which systemic imbalance and stress can be reduced by targeting ROS pathways. Unfortunately, this small body of evidence does not consider individuals with post-viral syndrome due to viral insult. For example, in one study conducted on generally healthy subjects, a plant-based broad-spectrum antioxidant dietary supplement blend (“PB-Blend”) was reported to exert significant reductive effects on real-time measures of ROS while simultaneously increasing NOHb. Boris V. Nemzer, et. al., (2017): Oxidative Stress or Redox Signalling, DOI:10.1080/10715762.2017.1390228. Although intriguing, this study was focused on the effects of a botanical blend on the cellular metabolic index, i.e., internal ratios of NAD+/NADH. From this study with healthy individuals, it was found that by ingesting the blend, participants would exhibit mitochondrial and cellular ROS generation inhibition by between only 9.2-7.7%, inhibition of nicotinamide-dinucleotide-phosphate oxidase system-dependent generation of ROS by between 12.0-14.8%, between 9.5-44.5% inhibition of extracellular H₂O₂ formation, and between 13.4-17.6% inhibition of TNF-α. All of which resulted in only 1.7-2.3 times increase of bioavailable NO concentration.

In another study with healthy participants, researchers inquired as to the effect of a similar dietary antioxidant supplement on certain oxidative stress markers (OSM). Boris V. Nemzer, et. al., (2014): Food Science & Nutrition, DOI:10.1002/fsn3.178. By observing OSM the researchers were measuring ex-vivo intra- and extracellular formation of ROS, respiratory activity of blood cells, mitochondrial-dependent ROS formation, and respiratory activity. This study also followed healthy participants' ability to modulate ex vivo cellular inflammatory responses induced by stimulation with exogenous TNF-α, nitrosative stress, and changes in NOHb concentrations. Although interesting, the administration of the antioxidant supplement resulted in inhibition of mitochondrial and cellular ROS generation by only 17%, 3.5 times inhibition of extracellular NOX2-dependent ROS generation, but nearly complete inhibition of extracellular H₂O₂, and a significant increase in circulating levels of NO.

Alternatively, in obese individuals, upon administration of a propriety polyphenol-rich blend, participants exhibited a 33-53% increase in NOHb and a 54-75% decrease in mitochondrial ROS. Boris V. Nemzer, et. al., (2021): A . . . Study on the Effects of a Plant-Based Dietary Supplement, DOI:10.5539/jfr.v10n2p21. Although impressive, this study failed to consider any effect of the polyphenol-rich blend on additional ROS pathways beyond the mitochondrial ROS pathway.

Thus, even though various systems and methods of modulating immune responses are known in the art, all or almost all of them suffer from several drawbacks especially when it comes to combined targeting of three specific ROS pathways—mitochondrial, iNOS dependent, and NOX2—in individuals with post-viral syndrome. Therefore, there remains a need for improved compositions and methods of alleviating signs or symptoms after viral infection, and especially after SARS-CoV2 infection.

### SUMMARY OF THE INVENTION

The inventive subject matter is directed to various compositions and methods of alleviating signs or symptoms of post-viral syndrome, and particularly post-COVID syndrome, in which oral administration of a polyphenol-rich composition that preferably contains at least 50 wt % total catechins and at least 20 wt % total chlorogenic acids modulates multiple and distinct pathways that generate reactive oxygen species, leading to a reduction in inflammation, mitochondrial dysfunction, and/or oxidative nitrosative stress.

In one aspect of the inventive subject matter, the inventors contemplate a method of alleviating a sign or symptom of a post-viral syndrome (and especially post-COVID syndrome or post-viral fatigue syndrome) that includes a step of orally administering a polyphenol-rich composition that predominantly comprises a plurality of chemically distinct catechins and plurality of chemically distinct chlorogenic acids. Most typically, administration of the polyphenol-rich composition acutely modifies multiple and distinct pathways that generate reactive oxygen species, thereby alleviating the sign or symptom of the post-viral syndrome.

Preferably, alleviation of the sign or symptom of the post-viral syndrome is associated with partial or complete amelioration of physical and/or mental function as compared to a subject not having been administered the composition.

In some embodiments, the plurality of chemically distinct catechins and plurality of chemically distinct chlorogenic acids are found in one or more of a green coffee bean extract, a green tea extract, a turmeric extract, tart cherry or extract thereof, and a cruciferous plant or extract thereof For example, the polyphenol-rich composition may be prepared from at least one of a green coffee bean extract, a green tea extract, a turmeric extract, a tart cherry or extract thereof, a broccoli or extract thereof, and a kale or extract thereof. Alternatively, or additionally, the polyphenol-rich composition may be prepared from at least two of a green coffee bean extract, a green tea extract, a turmeric extract, a tart cherry or extract thereof, a broccoli or extract thereof, and a kale or extract thereof. Therefore, and viewed from a different perspective, the polyphenol-rich composition will preferably include at least 50 wt % total catechins and at least 20 wt % total chlorogenic acids. In other embodiments, the polyphenol-rich composition may further comprise up to 30 wt % total supplemental antioxidants. For example, at least one of the supplemental antioxidants may be selected from the group consisting of a stilbenoid, curcumin, vitamin E, manganese, and coenzyme Q10. It is further generally contemplated that 10-150 mg of the polyphenol-rich composition are administered in a single dose (e.g., as a solid composition in a capsule) for a period of between 1 and 60 days, and is administered for at least 7 days after a negative viral test

After administration, alleviation of the sign or symptom of the post-viral syndrome will most typically occur within 30 minutes and 3 hours. As will be readily appreciated, the polyphenol-rich composition may be formulated in a capsule, a tablet, a gummy, a chewable, a dissolvable film, a powder or as a ready-to-drink beverage, a juice beverage, a carbonated beverage, or a liquid concentrate.

Preferably, the post-viral syndrome is post-COVID syndrome or post-viral fatigue syndrome.

In further embodiments of the inventive subject matter, the sign or symptom is fatigue, mitochondrial dysfunction, endothelial dysfunction, immune dysfunction, oxidative stress, chronic subacute inflammation, neurological dysfunction, and/or cognitive dysfunction. For example, contemplated pathways include mitochondrial reactive oxygen species generation, NOX2 dependent reactive oxygen species generation, and iNOS dependent reactive oxygen species generation. Additionally, administration of the polyphenol-rich composition may also acutely increase bioavailable NO, and/or acutely decrease proinflammatory cytokines (e.g., circulating IL1-beta, IL8, IL10, and/or TNF-alpha). In some embodiments, mitochondrial reactive oxygen species levels are reduced by at least 50%, iNOS activity was reduced by at least 55%, NOX2-dependent reactive oxygen species (ROS) generation was reduced by at least 40%, and/or circulating nitric oxide as measured by HbNO was increased by at least 30%.

Consequently, the inventors contemplate a polyphenol-rich composition for treatment of a sign or symptom of a post-viral syndrome, wherein the composition predominantly comprises a plurality of chemically distinct catechins and plurality of chemically distinct chlorogenic acids, and wherein the composition, upon oral administration at a dosage of between 10-150 mg, acutely modifies multiple and distinct pathways that generate reactive oxygen species, thereby alleviating the sign or symptom of the post-viral syndrome.

For example, the polyphenol-rich composition may include at least 50 wt % total catechins, at least 20 wt % total chlorogenic acids and optionally up to 30 wt % total supplemental antioxidants. Where desired, the polyphenol-rich composition may be prepared from at least one of a green coffee bean extract, a green tea extract, a turmeric extract, a tart cherry or extract thereof, a broccoli or extract thereof, and a kale or extract thereof. Among other suitable dosage forms, 10-150 mg of the polyphenol-rich composition may be prepared in a solid form and formulated into a capsule or tablet that is administered in a single dose daily for a period of between 1 and 60 days. Upon administration, the sign or symptom of post-viral syndrome may be alleviated within 30 minutes and 3 hours. In other embodiments, the polyphenol-rich composition may be formulated in a capsule, a tablet, a gummy, a chewable, a dissolvable film, or a powder. Among other formulations, the polyphenol-rich composition may also be formulated as a ready-to-drink beverage, a juice beverage, a carbonated beverage, or a liquid concentrate.

As noted above, contemplated post-viral syndromes include post-COVID syndrome and post-viral fatigue syndrome. For example, aches, pains, fever, upper and/or lower respiratory discomforts, shortness of breath, dyspnea, sore throat, fatigue, headache, abnormal chest imaging, and/or general malaise. Contemplated signs or symptoms may further include mitochondrial dysfunction, endothelial dysfunction, immune dysfunction, oxidative stress, chronic subacute inflammation, neurological dysfunction, and/or cognitive dysfunction. As such, observable markers for modification of these ROS pathways may be associated with inflammation (e.g., an interleukin or pro-inflammatory cytokine), may be associated with mitochondrial function (e.g., mitochondrial reactive oxygen species (ROS)), and/or may be associated with oxidative nitrosative stress (e.g., due to iNOS activity, NOX2-dependent ROS generation, etc.).

Viewed from another perspective, the inventors contemplate the use of a polyphenol-rich composition for the manufacture of a medicament for treatment of a sign or symptom of a post-viral syndrome, wherein the composition predominantly comprises a plurality of chemically distinct catechins and plurality of chemically distinct chlorogenic acids, and wherein the composition is formulated for administration at a dosage of between 10-150 mg.

In other words, the inventors contemplate a polyphenol-rich composition for use as a medicament in the treatment of post-viral syndrome, wherein the composition predominantly comprises a plurality of chemically distinct catechins and plurality of chemically distinct chlorogenic acids, and wherein the composition is formulated for administration at a dosage of between 10-150 mg.

Most typically, the polyphenol-rich composition acutely modifies multiple and distinct pathways that generate reactive oxygen species. Consequently, acute modification of the multiple and distinct pathways may alleviate aches, pains, fever, upper and/or lower respiratory discomforts, sore throat, fatigue, and/or general malaise typically associated with post-viral syndrome. The sign or symptom of a post-viral syndrome may further include mitochondrial dysfunction, endothelial dysfunction, immune dysfunction, oxidative stress, chronic subacute inflammation, neurological dysfunction, and/or cognitive dysfunction.

In some embodiments, the pathways include mitochondrial reactive oxygen species generation, NOX2 dependent reactive oxygen species generation, and/or iNOS dependent reactive oxygen species generation.

Preferably, the polyphenol-rich composition is provided in the form of plant materials and/or extracts thereof. For example, the plant materials may be selected from the group consisting of a green coffee bean extract, a green tea extract, a turmeric extract, a tart cherry or extract thereof, a broccoli or extract thereof, and a kale or extract thereof. In some embodiments, the polyphenol-rich composition may include at least 50 wt % total catechins, at least 20 wt % total chlorogenic acids, and optionally up to 30 wt % total supplemental antioxidants.

Consequently, administration of the polyphenol-rich composition may acutely increase bioavailable NO, and/or acutely decrease proinflammatory cytokines and/or chemokines, and/or acutely reduce circulating IL1-beta, IL8, IL10, and/or TNF-alpha. Such that mitochondrial reactive oxygen species levels are reduced by at least 50%, iNOS activity is reduced by at least 55%, wherein the NOX2-dependent ROS generation is reduced by at least 40%, and/or wherein the circulating nitric oxide as measured by NOHb is increased by at least 30%.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a graph depicting exemplary results for changes to cellular metabolic activity after administration of vitamin C and an exemplary polyphenol rich blend according to the inventive subject matter.

FIG. 2 is a graph depicting exemplary results for changes to iNOS dependent cellular metabolic activity after administration of vitamin C and an exemplary polyphenol rich blend according to the inventive subject matter.

FIG. 3 is a graph depicting exemplary results for changes to NOX2 dependent cellular metabolic activity after administration of vitamin C and an exemplary polyphenol rich blend according to the inventive subject matter.

FIG. 4 is a graph depicting exemplary results for changes to circulating NOHb after administration of vitamin C and an exemplary polyphenol rich blend according to the inventive subject matter.

FIG. 5A is a graph depicting exemplary results for changes to H₂O₂ formation, when TNF-αlevels simulate effects comparable to acute viral infection, after administration of vitamin C and an exemplary polyphenol rich blend according to the inventive subject matter.

FIG. 5B is a graph depicting exemplary results for changes to H₂O₂ formation, when TNF-α levels simulate a “Cytokine Storm,” after administration of vitamin C and an exemplary polyphenol rich blend according to the inventive subject matter.

### DETAILED DESCRIPTION

The inventors have discovered that certain polyphenol-rich blends, and especially blends that contain high concentrations of chemically distinct catechins and chemically distinct chlorogenic acids, can be used to modulate, and particularly downregulate, multiple and distinct pathways that generate reactive oxygen species and thereby alleviate signs and/or symptoms of post-viral syndrome (and especially post-COVID syndrome). Moreover, the compositions presented herein have also shown a stimulatory effect on bioavailable nitric oxide.

Numerous studies have previously established that viral infections can cause multiple complications to inflammatory, cardiovascular, and immune systems. Notably, infection with SARS-CoV2 is known to result in long-term effects, including changes in the lung, circulatory, renal and nervous systems. These effects are driven, in part, by acute and significant increases in multiple ROS species that are often associated with an exacerbated immune response and metabolic disturbance. In addition, post-viral syndrome has also been associated with vascular dysfunction, possibly due to low levels of bioavailable nitric oxide. On this backdrop the inventors investigated if, in subjects that were recovering from a moderate course of COVID-19, a polyphenol-rich blend could be employed to dampen multiple pathways leading to ROS generation and to increase bioavailable nitric oxide. In particular, the inventors focused on compositions that were able to modulate enzymatic systems that participate in the elevation of certain ROS which, if left uncontrolled, could cause metabolic dysfunction and reduction-oxidation reaction (redox) imbalances.

In one exemplary use, the inventors prepared a botanical blend that was characterized in a relatively high content of polyphenols, and particularly of chemically distinct catechins and chlorogenic acids. This exemplary blend had a total catechin content of more than 50 wt % and a total chlorogenic acid content of more than 20 wt % and was substantially free of antioxidant vitamins C and E. This exemplary blend contains green coffee bean extract, a green tea extract, a turmeric extract, tart cherry powder, broccoli powder, and kale powder, and 50 mg of the blend was filled into gelatin capsules to allow for simple oral administration. Administration of one or more such capsules post viral infection is then over a period of at least 14 days to achieve and/or sustain alleviation of at least one sign or symptom of post-viral syndrome, and especially post-COVID syndrome.

While the above composition is particularly preferred, it should be appreciated that numerous alternative compositions are also deemed suitable for use herein so long as such compositions have a polyphenol content in a dosage unit of at least 50%, and more preferably at least 60%, and most preferably at least 70% of the RDA. Viewed from a different perspective, contemplated compositions will provide, per administered dosage unit, an amount of total polyphenols of at least 20 mg, or at least 25 mg, or at least 30 mg, or at least 35 mg, or at least 40 mg. While typical dosage units will contain a polyphenol-rich composition in the range of between 25 and 500 mg, lower quantities (e.g., 10 mg, 20 mg) or higher quantities (e.g., 750 mg or 1,000 mg) are also deemed suitable for use herein, especially where such dosage units contain further active ingredients.

Furthermore, it is contemplated that suitable compositions will predominantly comprise a number of chemically distinct catechins and chemically distinct chlorogenic acids. Preferably, the contemplated composition will comprise at least 20 wt %, at least 30 wt %, or at least 45 wt % total chlorogenic acids. As such, the nature of the particular ingredients may vary to at least some extent. However, while it is preferred that the source materials for chlorogenic acids will comprise a coffee fruit or coffee bean derived material (e.g., extract from green beans and/or whole coffee fruit), other plant materials may also be used and include tea leaves, apple, pear, carrot, tomato, prune, bay leaf, mustard, celery, chives, basil, rosemary, sage, oregano, collard green, chicory, artichoke, burdock, eggplant, grape, kiwi, honeysuckle, blueberry, sunflower seed, zucchini, broccoli, cauliflower, arugula, asparagus, onion, spinach, peas, green pepper, okra, cabbage, and sweet potato. Most typically, such materials will be subjected to one or more isolation steps to enhance the concentration of the chlorogenic acids. In still further contemplated aspects, the chlorogenic acids may also be synthetic or semi-synthetic materials. Thus, contemplated chlorogenic acids include various esters of caffeic acid and quinic acid and their various isomers such as 5-O-caffeoylquinic acid (5-CQA), 4-O-caffeoylquinic acid (4-CQA), and 3-O-caffeoylquinic acid (3-CQA). Additionally contemplated chlorogenic acids include 3-O-p-Coumaroylquinic acid, 3-O-Dimethoxycinnamoylquinic acid, 3-O-Sinapoylquinic acid, 3,4-Di-O-caffeoylquinic acid, 3,4-Di-O-p-coumaroylquinic acid, 3-O-Caffeoyl-4-O-feruloylquinic acid, and their respective isomers at the 4 and 5 position.

Similarly, with respect to suitable catechins it is contemplated that the particular source of the catechins may vary considerably. Nonetheless, it is preferable for the contemplated composition to comprise at least 50 wt %, at least 60 wt %, or at least 75 wt % total catechins. However, it is generally preferred that the catechins are obtained from one or more plant materials, and particularly preferred plant materials include tea leaves (fresh, fermented, or otherwise extracted), cherry, broccoli, kale, celery, fava bean, green bean apple, blackberry, raspberry, apricot, black grape, pear, strawberry, guava, kale, cocoa etc. Most typically, such plant materials may be used in a raw state, dried state, fresh or fermented, or may be subjected to one or more isolation steps to enhance the concentration of the catechins. In still further contemplated aspects, the catechins may also be synthetic or semi-synthetic materials. Thus, contemplated catechins include C ((−)-catechin), EC ((−)-epicatechin), ECG ((−)-epicatechingallate), EGC ((−)-epigallocatechin), EGCG ((−)-epigallocatechin gallate), GC ((−)-gallocatechin), CG ((−)-catechingallate), and GCG ((−)-gallocatechingallate), as well as their various isomeric forms.

In still further contemplated aspects, the ratio of the chemically distinct catechins to the chemically distinct chlorogenic acids is preferably such that the catechins are present in higher quantities than the chlorogenic acids. For example, suitable ratios of catechins to chlorogenic acids include 10:1, 10:2, 10:3, 10:4, 10:5, 10:6, 10:7, 10:8, and 10:9, and in some embodiments, the catechins and the chlorogenic acids will be about the same weight proportions. Likewise, in less preferred (but still contemplated) aspects, the ratios of chlorogenic acids to catechins may be 10:1, 10:2, 10:3, 10:4, 10:5, 10:6, 10:7, 10:8, or 10:9.

Contemplated compositions may further include at least 2 wt %, at least 5 wt %, at least 10 wt %, or at least 15 wt %, of total supplemental antioxidants. Where desired, contemplated compositions may include between 2.5 wt % and 7.5 wt %, between 10 wt % and 20 wt %, or between 15 wt % and 30 wt %. As will be readily appreciated, suitable amounts of supplemental antioxidants may also include up to 10 wt %, up to 20 wt %, up to 30 wt %, or up to 40 wt %. In some embodiments, preferred antioxidants include stilbenoids such as resveratrol or any derivatives, piceatannol, and/or trans-4-hydroxystilbene. Alternatively, or additionally, supplemental antioxidants also include curcumin, coenzyme Q10, thiamine, riboflavin, nicotinic acid, folic acid, creatine, L-arginine, glutathione, α-lipoic acid vitamin A, vitamin C, vitamin E, and/or minerals including selenium, copper, zinc, and manganese. In some embodiments, a supplemental antioxidant may be included alone or in combination with at least one other supplemental antioxidant of a different variant to constitute between 10% and 50% of the RDA. For example, curcumin and/or resveratrol may be present in contemplated compositions in quantities of equal or less than 10 wt %, or equal or less than 8 wt %, or equal or less than 6 wt %, or equal or less than 4 wt % of the total wt %. Viewed from a different perspective, the quantity of curcumin and/or resveratrol in a single dosage unit will typically be equal or less than 10 mg, or equal or less than 8 mg, or equal or less than 6 mg, or equal or less than 4 mg. When in combination, supplemental antioxidants need not be included in equal amounts, and while present in some embodiments need not be present in other embodiments. Supplemental antioxidants can be added and/or removed depending on the antioxidant pathway that is being targeted.

Consequently, it should be recognized that contemplated compositions may comprise a number of distinct classes of antioxidants, which will typically also have distinct underlying mechanisms of action. For example, some antioxidants may act as direct redox agent, while other antioxidants will function as an inhibitor of one or more ROS generating enzymes, while still other antioxidants will function as inhibitors of proinflammatory mediators that are involved in downstream cell-based ROS generation. Moreover, and especially where the source of the catechins and/or the source of chlorogenic acids is a part of the coffee plant or tea plant, it should be appreciated that contemplated compositions may also include appreciable quantities of caffeine, typically below 5% of the composition (e.g., equal or less than 10 mg, or less than 5 mg per dosage unit).

Regardless of the type and quantity of the multiple ingredients, it is generally contemplated that the polyphenol-rich composition will be administered to a subject suffering from a sign or symptom of a post-viral syndrome at least once daily using a dosage unit of between 10 mg and 500 mg over a period of at least two weeks. For example, a subject can take 50 mg once daily over a period of two weeks. However, in alternative embodiments, the composition can be taken twice or three times daily, for example, using a dosage unit of 10 mg or 25 mg, or 50 mg. Moreover, it should be appreciated that the duration of administration may be less than two weeks or significantly longer. For example, a subject can also take 10 mg once daily over a period of 60 days or 100 mg once daily over a period of 5 days. Most typically, resolution of the signs and symptoms will be a guide for subjective duration. In some embodiments, signs and/or symptoms will be resolved in at least 15 minutes, at least 30 minutes, at least 1 hour, at least 1.5 hours, at least 3 hours, or at least 4 hours after administration. Among other signs and symptom, especially contemplated signs and symptoms include fatigue, malaise, headache, dyspnea, diarrhea, low blood nitric oxide levels, redox imbalance/oxidative stress, aches, pains, fever, upper and/or lower respiratory discomforts, shortness of breath, sore throat, abnormal chest imaging, loss of memory, “brain fog,” mental confusion, etc.

As should be readily appreciated, while contemplated compositions are preferably administered orally using a capsule, other modes of administration and formulations are also deemed suitable herein and include infusion or injection, and more commonly oral administration in liquid or solid form commonly used with nutritional supplements. Among other choices, administrable forms include a capsule, a table, a gummy, a chewable, a dissolvable film a powder, a ready-to-drink beverage, a juice beverage, a carbonated beverage, or a liquid concentrate.

While not wishing to be bound by any specific theory or hypothesis, it is contemplated that the compositions will affect multiple enzymatic systems in vivo, and particularly mitochondrial, NOX2, and iNOS enzymatic systems, that are dysregulated in the wake of a viral infection as is shown in more detail below. Moreover, contemplated compositions will also advantageously increase bioavailable nitric oxide (in the form of nitrosylated hemoglobin, (NOHb)), which is also found to be suppressed in subjects after viral infection. Consequently, contemplated compositions are deemed particularly advantageous to alleviate or reduce signs and/or symptoms of a post-viral syndrome, and particularly post-COVID syndrome. With respect to the viral infection, it should also be noted that while SARS-CoV2 and its subvariants are especially contemplated, other viruses are also deemed suitable and include all viruses for which post-viral syndrome is known or suspected (e.g., influenza, MERS, Epstein-Barr virus, cytomegalovirus, human herpesvirus, enteroviruses, rhinoviruses, etc.).

### EXAMPLES

A randomized, double-blind study was performed on 28 individuals 18-24 days after a moderate COVID-19 infection (CDC definition of moderate course and return to work criteria as noted at URL: www.cdc.gov/coronavirus/2019-ncov/hcp/return-to-work.html). All subjects received a single dose of 50 mg of an exemplary polyphenol-rich blend or 1,000 mg vitamin C. Real-time cellular formation of ROS was measured using a portable electron spin resonance (ESR) spectrometer to identify changes in levels of bioavailable NO (measured as circulating NOHb), formation of mitochondrial, NOX2-, iNOS-, and TNFα-dependent ROS generation before, and then at 30, 60, 120, and 180 minutes following administration. Inflammatory, immunity (hsCRP, TNF-alpha plasma level), interleukin (IL-1, IL6, IL8, IL10), cytokine (IFN-Y, TNF-α, NF-kB), and immunoglobulin (IgA, IgM, IgG, IgE) profiles were also followed. In addition to laboratory and cell function investigations, the inventors performed clinical cardio ergometry, blood O₂ saturation, and respirometry examinations. Unless indicated otherwise, tests were performed using methods and devices as described in WO 2018/192635, and cytokine and interleukin concentrations were determined using commercially available standard test kits.

Notably, and as is shown in more detail below, the collected baseline data revealed that mitochondrial, NOX2, and iNOS enzymatic systems were significantly involved in the generation of ROS 18-24 days after a positive COVID-19 PCR test. Remarkably, a single dose administration of the exemplary polyphenol-rich blend as described above had a multifunctional impact on ROS species, significantly inhibiting: (1) mitochondrial ROS levels by up to 56%, (2) iNOS by up to 60%, and (3) NOX2-dependent ROS generation by up to 49%. Vitamin C also exhibited ROS-mitigating activity, however, more narrowly and selectively inhibiting NOX2-dependent ROS generation by 45%. In addition, circulating NOHb levels were also significantly increased (33%) after administration of the exemplary polyphenol-rich blend, but not after administration of vitamin C. The exemplary polyphenol-rich blend and vitamin C exhibited equal potential to reduce high dose TNFα (200 ng/ml)-induced H₂O₂ formation. Still further, the influence of the exemplary polyphenol-rich blend on redox imbalance positively and acutely improved selected interleukins (IL1beta, IL8, IL10) and cytokine (TNFα) plasma concentrations after 3h of single-dose administration.

Study design: Pilot, exploratory, double-blind, randomized study for single-dose administration of VDF products after moderate COVID-19 disease course.

Study duration: 2-3 weeks.

Population: 13 male and 15 female volunteers (see inclusion and exclusion criteria) randomized into 2 parallel groups for administration of a single dose of 50 mg of the polyphenol-rich blend or 1000 mg Vitamin C capsules.

Randomization: Draw each male subject's number, one at a time, from the Subjects container and immediately draw one scrap of paper from the Treatments container. By this action, each subject was randomly assigned their treatment. The same was performed for female subjects' numbers.

Blinding: Identically sized capsules containing study materials were prepared by study sponsor and placed into bottles labelled with either “A” or “B”. In the following, Group A denotes subjects that were administered 50 mg of the exemplary polyphenol-rich blend (blend of green coffee bean extract, green tea extract, turmeric extract, tart cherry powder, broccoli powder, and kale powder, containing at least 50 wt % total catechins and at least 20 wt % total chlorogenic acids) while Group B denotes subjects that were administered 1,000 mg of vitamin C. Study provider administered the treatments accordingly as described in the randomization procedure. Study provider did not receive the blinding “key” until all raw data had been completed and delivered. A third party, also unaware of the key, conducted independent statistical analyses of the raw data.

Definition of moderate COVID-19 infection: Confirmation of COVID-19 infection (positive initial PCR COVID-19 test) and meeting CDC criteria for moderate disease course. Individuals who had any of the various signs and symptoms of COVID 19 (e.g., fever, cough, sore throat, malaise, headache, muscle pain, shortness of breath, dyspnea, or abnormal chest imaging) and evidence of lower respiratory disease by clinical assessment or imaging and a saturation of oxygen (SpO2)≥94% on room air at sea level.

Inclusion criteria: a. Confirmed Covid-19 infection; b. Negative Covid-19 quick test on day of examination; c. CDC criteria for return to work are met: At least 10 days and up to 20 days have passed since symptoms first appeared, and at least 24 hours have passed since last fever without the use of fever-reducing medications, and symptoms (e.g., cough, shortness of breath) have improved; d. Otherwise medically stable population; e. BMI between 24-30; and f. Age between 40- and 55-years.

Exclusion criteria: No intake of vitamins/supplements 2 weeks before inclusion. No medication known to affect endothelial function. Generally accepted contraindications to physical exercise; smokers, type 1 and type 2 diabetes; liver and kidney impairments; psychiatric disorders, other disorders of acute or chronic nature (gastrointestinal, pulmonary, renal, cardiac, neurological, or psychiatric disorders), use of weight-reducing preparations or appetite suppressants, participation in a clinical study within the last 30 days before the beginning of this study or during this study. Health status was checked by clinical and laboratory examination.

Assessment of oxidative nitrosative stress level included (i) Total/cellular ROS (intra-/extracellular ROS generation), (ii) Mitochondrial dependent ROS formation (mitochondrial dysfunction), (iii) NOX2 dependent ROS formation (phagocytic/infl. NADPH-oxidase), (iv) iNOS dependent ROS formation (inducible/infl. iNOS dysfunction), and (v) Vit. C cellular depletion assay (Vit. C cellular depletion).

In addition, routine blood tests were conducted and included a haemogram, kidney function test (creatinine, urea) and a liver function test (ALT, AST, GGT, bilirubin, ALP). Metabolic status was determined by fasting glucose levels (for metabolic syndrome evaluation), plasma insulin (for metabolic syndrome evaluation), insulin tolerance index (for metabolic syndrome evaluation), and HbAlc (for prediabetes/diabetes evaluation).

Inflammatory/Immunity profile was determined by quantitating hsCRP, TNF-alpha plasma level (as early indicator of inflammation), Interleukins (IL-1, IL6, IL8, IL10), Cytokines (titer of IFN-Y, TNF-α, NF-kB), Immunoglobulin (titer of IgA, IgM, IgG, IgE) profile. A TNF-αhypersensitivity assay was performed for evaluation of inflammatory resistance (robustness to “Cytokine Storm”).

A cellular metabolic activity (CMA) and extended cellular metabolic activity (eCMA) assay was used for the real-time monitoring of cellular, mitochondrial. NADPH oxidase 1 or 2, peroxidase-, and inducible-nitric-oxide-synthase-dependent (iNOS-dependent) generation of reactive oxygen species. Cellular membrane and mitochondria permeable spin probe 1-hydroxy-3-methoxycarbonyl-2.2.5.5-tetramethylpyrrolidine (CMH, 1 mM) dissolved in KHB-buffer (20 mM; pH 7.4) was mixed with freshly drawn capillary blood to perform measurements of ROS generation under controlled temperature and oxygen concentration (t=36.6° C., pO2=110 mm/Hg) (Nemzer et al. 2014). For the extended CMA (eCMA) analysis, a portion of the sample was taken and kept in ice bath samples at 4° C. It was mixed with: a.) superoxide dismutase (SOD, 50 mU/ml; eCMA-ENDO) to measure amounts of extracellularly released O2- by NOX1; b.) catalase (50 mU/ml, eCMA-INFLA) to analyze peroxidase-dependent H2O2 formation; c.) antimycin A (10 μM, eCMA-MITO) to evaluate mitochondria-dependent O2-/H2O2 generation; d.) Apocynin (10 μM, eCMA-PHAGO-NOX2) to detect phagocytic NADPH-dependent (NOX2-dependent) O2-formation; and, e.) 1400W (0.1 μM, eCMA-iNOS) to identify inducible-nitric-oxide-dependent (iNOS-dependent) O2-/ONOO-generation. The addition of oxygen label (NOX-15.1, 5 μM) to the blood sample made it possible to monitor the oxygen concentration of cellular, mitochondrial, NADPH oxidase-, and peroxidase-dependent oxygen consumption. The ESR signal was detected using the NOXYGEN System and in parallel with a portable VitaScreen ESR spectrometer. Calibration of the ESR signal was performed using a calibration solution with a standard concentration of CP° (500 μM) or oxygen label (NOX-15.1, 100 μM) filled into oxygen-permeable 50 μl PTX capillary by deoxygenation of oxygen label solution using perfusion of pure nitrogen (99.99%).

Inflammatory resistance was measured using an inflammatory resistance assay. The ex vivo assay describes changes in extracellular H₂O₂ generation by blood cells after a) exposure to TNF-α at a final concentration of 40 ng/ml, representative of elevated TNF-α plasma concentrations in human blood, and then b) exposure to 200 ng/ml (a 5-times higher amount of TNF-α and comparable to the amount of TNF-α that could be observed in a person infected with COVID-19) in order to mimic the conditions of a cytokine storm.

To measure circulating levels of NOHb, a bioavailable NO concentration assay was used where heparinized venous blood samples, that were previously quick frozen in liquid nitrogen and stored at −80° C., were analyzed for NOHb content at −196° C. in a quartz finger Dewar. The ESR spectrometer NOXYSCAN SYSTEM, equipped with a specially designed cavity, was operating at 100 kHz field modulation to collect the ESR spectra at X-band 9.7 GHz using the following settings: microwave power: 50 mW; modulation amplitude: 8 G; center field: 2.01 g; sweep width: 60 G; conversion time: 20 ms; time constant: 80 ms; number of scans: 60; total detection time: 600 seconds. The amount of detected NO· was determined from the calibration curve for the intensity of the ESR signal of erythrocytes treated with known concentrations of nitrite (1-25 μM) and Na2S2O4 (20 mM).

To further investigate the role of cytokines and interleukins in a subject's immune response during viral infection, the inventors performed profiling of inflammatory markers such as IL16, IL6, IL8, IL10, IFNγ, and TNFα. Inflammatory markers were measured using electrochemiluminescence-based V-Plex immunoassays (MesoScale Discovery, Gaithersburg, MD, USA) in plasma from samples collected before and after single-dose supplementation of PB-Blend or Vitamin C.

Chemicals used for the spin probes: 1-hydroxy-3-methoxycarbonyl-2.2.5.5-tetramethylpyrrolidine (CMH, NOX-02.5-VIT), 1-hydroxy-4-phosphono-oxy-2.2.6, 6-tetramethylpiperidine (PPH, NOX-03.2); for the ESR: Krebs HEPES Puffer VIT (KHB-VIT, NOX-21.2-VIT) containing metal chelators deferoxamine (DF, NOX-09.1) and diethyldithiocarbamate (DETC, NOX-10.1), Heparin (100 U/ml) and Krebs-Hepes buffer (KHB, NOX-07.6); oxygen label (NOX-15.1); and for the eCMA working solutions: eCMA MITO (NOX-22.1-VIT), eCMA ENDO (NOX-23.1-VIT), eCMA-INFLA (NOX-24.1-VIT), eCMA-iNOS (NOX-26.1-VIT), eCMA PHAGO NOX2 (NOX-25.1-VIT) were obtained from Noxygen Science Transfer & Diagnostics (Elzach, Germany). All other chemicals and reagents used were of analytical grade and purchased from Sigma-Aldrich (St. Louis, MO, USA) unless otherwise specified.

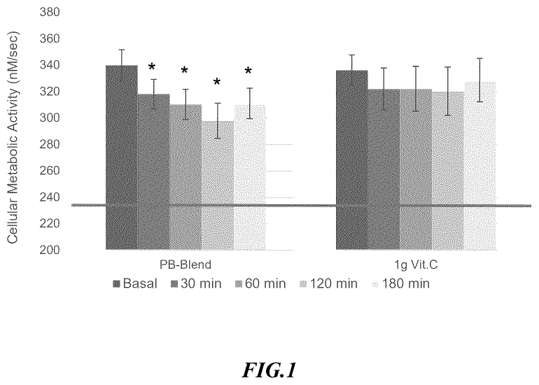

As can be seen from FIG. 1, cellular metabolic activity was at base line above levels considered healthy as indicated by the blue line. Administration of the exemplary polyphenol-rich blend resulted in a significant acute reduction in cellular metabolic activity over three hours, while administration of vitamin C reduced cellular activity to some degree in the same time frame (albeit not in a statistically significant manner).

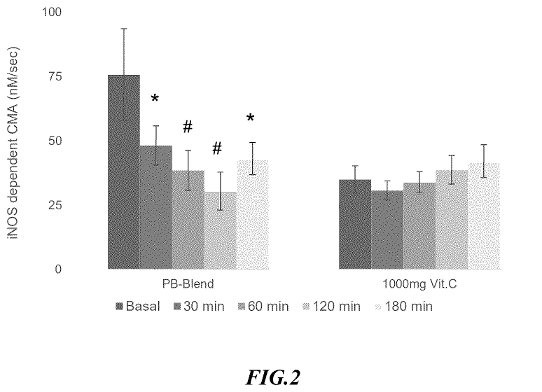

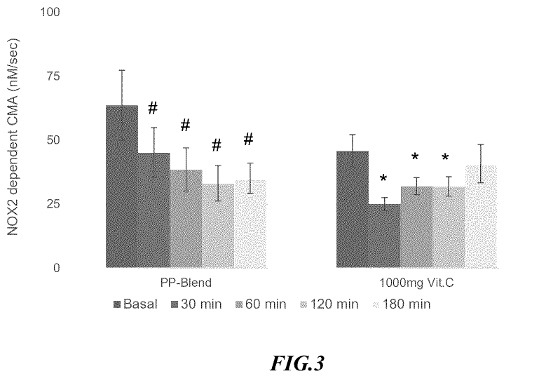

With regard to iNOS dependent cellular metabolic activity, it was once more observed that administration of the exemplary polyphenol-rich blend significantly and acutely reduced exacerbated metabolic activity while at the same time frame vitamin C had no notable effect as is shown in FIG. 2. With respect to NOX2 dependent cellular metabolic activity as shown in FIG. 3, the exemplary polyphenol-rich blend both reduced cellular metabolic activity and maintained reduced levels of NOX2 dependent cellular metabolic activity as compared with administration of vitamin C.

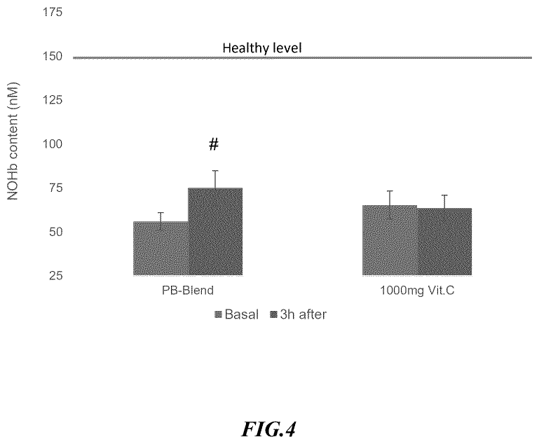

Additionally, the inventors demonstrated that the exemplary polyphenol-rich blend also had a beneficial effect on biologically available nitric oxide by way of determination of circulating nitrosylated hemoglobin (NOHb). Comparative results for the exemplary polyphenol-rich blend and vitamin C are shown in FIG. 4.

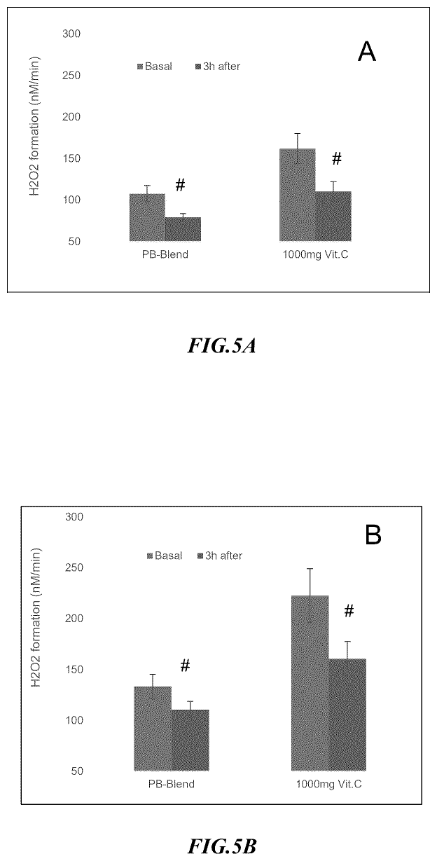

With regard to TNF-α induced H₂O₂ formation, the inventors demonstrated that after simulation with TNF-α (40 ng/ml, comparable to acute viral infection), both the exemplary polyphenol-rich blend and vitamin C could similarly suppress the generation of extracellular H₂O₂ induced by TNF-α as shown in FIG. 5A. Likewise, with a higher TNF-α concentration (200 ng/ml) to simulate a “Cytokine Storm,” the inhibitory capacity of both the exemplary polyphenol-rich blend and vitamin C were again similar as shown in FIG. 5B. However, it should be noted that vitamin C was administered at a 20-fold higher amount as compared to the exemplary polyphenol-rich blend.

Further statistical analyses were performed using repeated measures ANOVAs (rm-ANOVA). Statistical rm-ANOVA tests require the data to be normally distributed. As such, the data was first examined for outliers within each variable of interest. Outliers were determined using a Tukey fences approach ((Q₁−1.5*(IQR)) and (Q₃+1.5*(IQR)), where Q₁ is the first quartile, and Q₃ is the third quartile). Outliers exceeding these boundaries were excluded from the analysis. Rm-ANOVAs were performed with the group as the between-subjects factors. In the case of interactions, univariate ANOVAs were performed to determine the nature of the interaction to establish appropriate inferences. Results are presented as the mean±standard error of the mean (SEM). Statistical significance was determined at the p<0.05 level.

Together, these results suggest that administration of at least 50 mg of the exemplary polyphenol-rich blend has significant potential to increase bioavailable NOHb and to modulate a broad spectrum of enzymatic systems related to mitochondria, iNOS, and NOX2-dependent ROS generation. The exemplary polyphenol-rich blend exhibits unique antioxidative activity that may support against uncontrolled ROS generation in subjects with imbalanced systems due to viral infection. Comparatively, administration of 1,000 mg vitamin C revealed a more narrowly focused antioxidative activity, limited only to inhibition of NOX2-dependent generation of ROS. Consequently, the exemplary polyphenol-rich blend can be considered a new alternative to, or in tandem with, vitamin C, or alternative supplemental antioxidants, in applications for oxidative balance, immune support, and recovery, especially for potential modulation of inflammatory responses to viral insult that drive feelings of sickness and malaise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1. A method of alleviating a sign or symptom of a post-viral syndrome, comprising:
orally administering a polyphenol-rich composition that predominantly comprises a plurality of chemically distinct catechins and plurality of chemically distinct chlorogenic acids; and
wherein administration of the polyphenol-rich composition acutely modifies multiple and distinct pathways that generate reactive oxygen species, thereby alleviating the sign or symptom of the post-viral syndrome.

2. The method of claim 1, wherein alleviating the sign or symptom of the post-viral syndrome is associated with partial or complete amelioration of physical and/or mental function as compared to a subject not having been administered the composition.

3. The method of claim 1, wherein the plurality of chemically distinct catechins and plurality of chemically distinct chlorogenic acids are present in one or more of a green coffee bean extract, a green tea extract, a turmeric extract, tart cherry or extract thereof, and a cruciferous plant or extract thereof.

4. (canceled)

5. (canceled)

6. The method of claim 1, wherein the polyphenol-rich composition comprises at least 50 wt % total catechins and at least 20 wt % total chlorogenic acids, and wherein the polyphenol-rich composition further comprises up to 30 wt % total supplemental antioxidants.

7. (canceled)

8. (canceled)

9. The method of claim 1, wherein 10-150 mg of the polyphenol-rich composition are administered in a single dose daily for a period of between 1 and 60 days, and/or wherein alleviating the sign or symptom of post-viral syndrome occurs within 30 minutes and 3 hours upon administration.

10. (canceled)

11. The method of claim 1, wherein the polyphenol-rich composition is formulated in a capsule, a tablet, a gummy, a chewable, a dissolvable film, or a powder, and/or wherein the polyphenol-rich composition is formulated as a ready-to-drink beverage, a juice beverage, a carbonated beverage, or a liquid concentrate.

12. (canceled)

13. The method of claim 1, wherein the post-viral syndrome is post-COVID syndrome or post-viral fatigue syndrome.

14. The method of claim 1, wherein the polyphenol-rich composition is administered for at least 7 days after negative viral test.

15. The method of claim 1, wherein the sign or symptom is fatigue, mitochondrial dysfunction, endothelial dysfunction, immune dysfunction, oxidative stress, chronic subacute inflammation, neurological dysfunction, and/or cognitive dysfunction.

16. The method of claim 1, wherein one of the pathways is mitochondrial reactive oxygen species generation, wherein one of the pathways is NOX2 dependent reactive oxygen species generation, and/or wherein one of the pathways is iNOS dependent reactive oxygen species generation.

17. (canceled)

18. (canceled)

19. The method of claim 1, wherein administration of the polyphenol-rich composition acutely increases bioavailable NO, and/or wherein administration of the polyphenol-rich composition acutely decreases proinflammatory cytokines.

20. The method of claim 1, wherein administration of the polyphenol-rich composition acutely reduces circulating IL1-beta, IL8, IL10, and/or TNF-alpha.

21. The method of claim 1, wherein mitochondrial reactive oxygen species levels are reduced by at least 50%, iNOS activity is reduced by at least 55%, wherein the NOX2-dependent ROS generation is reduced by at least 40%, and/or wherein the circulating nitric oxide as measured by NOHb is increased by at least 30%.

22. A polyphenol-rich composition for treatment of a sign or symptom of a post-viral syndrome, wherein the composition predominantly comprises a plurality of chemically distinct catechins and plurality of chemically distinct chlorogenic acids, and wherein the composition, upon oral administration at a dosage of between 10-150 mg, acutely modifies multiple and distinct pathways that generate reactive oxygen species, thereby alleviating the sign or symptom of the post-viral syndrome, optionally wherein alleviating the sign or symptom of post-viral syndrome occurs within 30 minutes and 3 hours upon administration.

23. The polyphenol-rich composition of claim 22, wherein the polyphenol-rich composition comprises at least 50 wt % total catechins, at least 20 wt % total chlorogenic acids, and optionally up to 30 wt % total supplemental antioxidants, and/or wherein the polyphenol-rich composition is prepared from at least one of a green coffee bean extract, a green tea extract, a turmeric extract, a tart cherry or extract thereof, a broccoli or extract thereof, and a kale or extract thereof.

24-28. (canceled)

29. The polyphenol-rich composition of claim 22, wherein the post-viral syndrome is post-COVID syndrome or post-viral fatigue syndrome.

30. The polyphenol-rich composition of claim 22, wherein the sign or symptom is aches, pains, fever, upper and/or lower respiratory discomforts, shortness of breath, dyspnea, sore throat, fatigue, headache, abnormal chest imaging, and/or general malaise.

31. The polyphenol-rich composition of claim 22, wherein the sign or symptom is mitochondrial dysfunction, endothelial dysfunction, immune dysfunction, oxidative stress, chronic subacute inflammation, neurological dysfunction, and/or cognitive dysfunction.

32. The polyphenol-rich composition of claim 22, wherein one of the pathways is mitochondrial reactive oxygen species generation, wherein one of the pathways is NOX2 dependent reactive oxygen species generation, and/or wherein one of the pathways is iNOS dependent reactive oxygen species generation, and optionally wherein mitochondrial reactive oxygen species levels are reduced by at least 50%, iNOS activity is reduced by at least 55%, wherein the NOX2-dependent ROS generation is reduced by at least 40%, and/or wherein the circulating nitric oxide as measured by NOHb is increased by at least 30%.

33. The polyphenol-rich composition of claim 22, wherein administration of the polyphenol-rich composition acutely increases bioavailable NO, and/or wherein administration of the polyphenol-rich composition acutely decreases proinflammatory cytokines.

34-57. (canceled)
